(19) United States
(12) Patent Application Publication
(10) Pub. No.: US 2023/0290457 A1
(43) Pub. Date: Sep. 14, 2023

(54) ARTIFICIAL INTELLIGENCE MEDICAL DEVICE

(71) Applicant: Biocogniv Inc., Burlington, VT (US)

(72) Inventors: Artur Borges Adib, Winooski, VT (US); Tanya Sharlene Kanigan, Shelburne, VT (US); Robert Aaron Levine, Portsmouth, NH (US); Steven Andrew Wallace, Hinesburg, VT (US); Bennett Eli Siegel, Burlington, VT (US)

(21) Appl. No.: 18/199,031

(22) Filed: May 18, 2023

Related U.S. Application Data
(63) Continuation-in-part of application No. 17/210,382, filed on Mar. 23, 2021, now Pat. No. 11,676,722.
(60) Provisional application No. 62/993,671, filed on Mar. 23, 2020.
(60) Provisional application No. 63/023,132, filed on May 11, 2020.

(51) Int. Cl. G16H 10/60; G16H 50/30; G16H 50/70

(52) U.S. Cl. CPC G16H 10/60; G16H 50/30; G16H 50/70

(57) ABSTRACT

A medical device server can include a plurality of artificial intelligence medical devices, each trained for rendering a prediction related to a disease or medical condition. A healthcare provider can obtain patient data, including medical tests results, and send them to the medical device server as a request to obtain disease or health condition predictions. The patient data can be provided to an applicable artificial intelligence medical device whose input fields data type match and are present in the patient data embedded in a request received from the healthcare provider. The applicable artificial intelligence medical devices can render disease or health condition predictions, which are communicated to the healthcare provider in response to the request.

### CROSS-REFERENCE TO RELATED APPLICATIONS

This application is a continuation in part of U.S. patent application Ser. No. 17/210,382, filed on Mar. 23, 2021, entitled “Method of Early Detection, Risk Stratification, and Outcomes Prediction of a Medical Disease or Condition with Machine Learning and Routinely Taken Patient Data,” which claims the benefit of priority of U.S. Provisional Application No. 62/993,671 filed on Mar. 23, 2020 and U.S. Provisional Application No. 63/023,132 filed on May 11, 2020, the contents of all of which are hereby incorporated by reference in their entirety and should be considered a part of this disclosure.

### BACKGROUND

### Field

This invention relates generally to the field of artificial intelligence (AI) models and their usage, and more particularly to AI models used in diagnosis and prediction of diseases or medical conditions.

### Description of the Related Art

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Advancements in the field of AI can provide substantial advantages in the field of healthcare. In particular, healthcare providers can benefit from AI medical devices that can assist in early detection of medical conditions, which can help them administer effective and preventative measures aimed at stopping or slowing down the course of potentially complicated health conditions. As a result, there is a need in the healthcare industry, for robust medical devices that take advantage of the advancements in the field of AI and provide efficient assistance to healthcare providers.

### SUMMARY

The appended claims may serve as a summary of this application.

### BRIEF DESCRIPTION OF THE DRAWINGS

These drawings and the associated description herein are provided to illustrate specific embodiments of the invention and are not intended to be limiting.

FIG. 1 illustrates an AI medical device environment, where the services of one or more AI medical devices can enhance the quality of patient care.

FIG. 2 illustrates a flowchart of a method of the operations of the embodiment of FIG. 1.

FIG. 3 illustrates a flowchart of an example method of the operations of a medical device server.

FIG. 4 illustrates a diagram of an embodiment, where a medical device server can use a validation module to improve the quality and accuracy of the predictions generated by an AI medical device.

FIG. 5 illustrates a flowchart of a method of training an AI model in an AI medical device.

FIG. 6 illustrates an environment in which some embodiments may operate.

### DETAILED DESCRIPTION

The following detailed description of certain embodiments presents various descriptions of specific embodiments of the invention. However, the invention can be embodied in a multitude of different ways as defined and covered by the claims. In this description, reference is made to the drawings where like reference numerals may indicate identical or functionally similar elements.

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail. When the terms “one”, “a” or “an” are used in the disclosure, they mean “at least one” or “one or more”, unless otherwise indicated.

Modern advancements in the field of Artificial intelligence (AI) have opened up a world of possibilities for various industries. The medical device industry is among the industries, which can benefit substantially from advancements in the AI. AI models can become software-based medical devices capable of enhancing patient care. For example, disease diagnosis using AI technology can greatly assist health practitioners. Consequently, AI models can be used as AI medical devices.

Furthermore, AI performance can be substantially enhanced by an increase in availability of good quality training data. When a patient visits a medical care facility, such as a hospital, particularly when the patient visits an urgent care, or an emergency room, the patient typically receives a number of laboratory orders and observations, depending on the symptoms the patient is presenting. The patient data collected across a plurality of medical facilities can be used to train and enhance the quality of AI medical devices. It can be advantageous to utilize the services of one or more AI medical devices to enhance the quality of patient care.

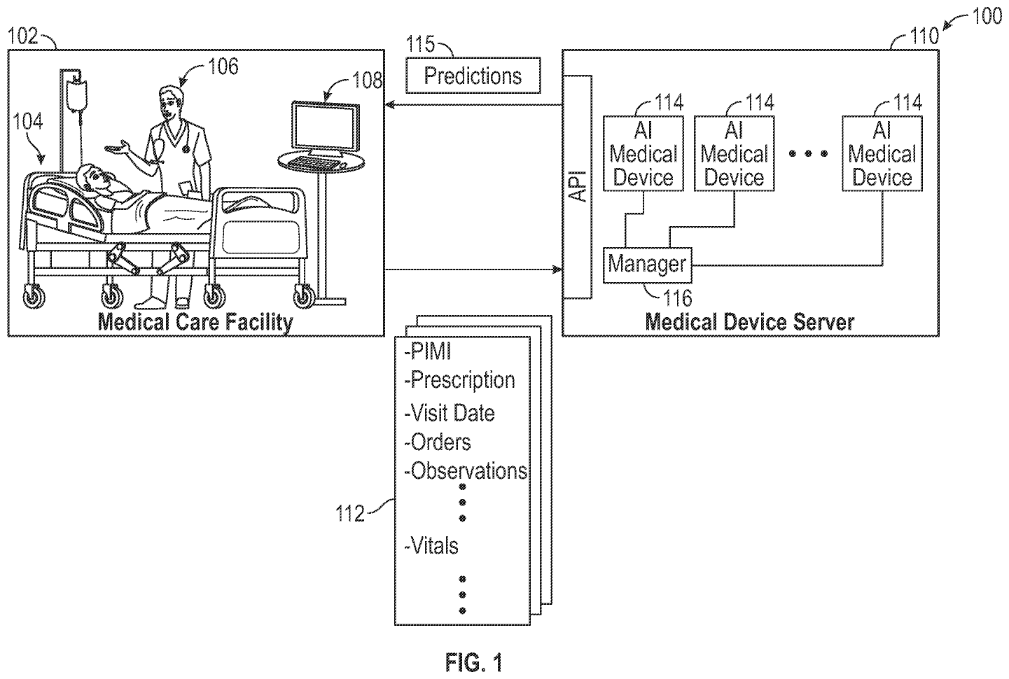

FIG. 1 illustrates an AI medical device environment 100, where the services of one or more AI medical devices 114 can enhance the quality of patient care. In some applications of the environment 100, AI medical devices can be used to diagnose or predict the presence or severity of a medical disease or condition. In particular, AI medical devices can prove beneficial in situations where the patient is developing a potentially life-threatening condition, such as sepsis. However, the described embodiments can be applied to deploy AI medical devices for a variety of diseases and medical conditions.

The environment 100 includes a medical care facility 102, such as a hospital. Patient 104 visits a health care provider 106. The provider 106 can use a terminal device 108 to be in communication with a medical device server 110. The medical device server 110 can be a collection of hardware and software components provided by an independent and external server, or it can be part of the internal computer or cloud infrastructure network of the medical care facility 102. Depending on the observed patient symptoms, the provider 106 can enter various laboratory orders for the patient 104 in the terminal 108. Example laboratory orders can include various blood tests, such as BMP, CBC with differential, and many others. The provider 106 can also obtain patient vitals, such as blood pressure, body temperature, blood oxygen level, etc. Other patient data, such as patient history, height, weight, and current prescribed medications can also be obtained, or otherwise retrieved from patient electronic medical record (EMR). The results of the laboratory orders can be termed “observations” and can be generated by the staff and/or equipment at the medical care facility 102.

While the embodiments will be described in terms of the use case where a patient 104 is observed or is otherwise physically present in a medical care facility 102, not all embodiments require this type of environment to operate. For example, a patient may be visited by a healthcare provider at home, where the healthcare provider can be in communication with various medical servers, including the medical device server 110. In some embodiments, patients 104 can be independently in communication with the medical device server 110 to use its services.

The medical device server 110 can have various software and hardware components, including communication modules, processor components and database components. In some embodiments, the medical device server 110 does not store any patient data; instead, it spawns an impermanent process when a request for health prediction is received. The process can receive patient data and operate on an impermanent memory, such as a random-access memory (RAM). Once the process is completed, the patient data is deleted from the impermanent memory. In other words, in some embodiments, the medical device server 110 can provide its services in a stateless manner, not preserving any previous patient data, other than what is provided to it in a live request. In other embodiments, the medical server 110 can include storage components and operate as a state machine, recalling previous patient data in relation to the patient data subsequently received.

The medical device server 110 can include one or more AI medical devices 114. Each AI medical device 114 can be an AI model trained for detecting a medical disease or condition. Examples include a sepsis AI medical device 114, an acute kidney injury (AKI) AI medical device 114, a COVID AI medical device 114 and many others. Furthermore, the AI medical devices 114 can be directed to selected age groups. For example, the medical device server 110 can include both adult sepsis AI medical device 114 and pediatric sepsis AI medical device 114 as separate AI medical devices.

The medical care facility 102 can send one or more health prediction requests 112 to the medical device server 110. In some embodiments, the medical device server 110 can receive the requests 112 via an application programming interface (API). The requests 112 can be sent automatically, for example, based on when a selection of patient health data becomes available. The requests 112 can also be sent manually, for example, by entering a command in the terminal device 108. In response to the requests 112, the medical device server 110, and/or the components therein, can return predictions 115 to the medical care facility 102. The provider 106 can review the predictions 115 (e.g., on the terminal 108) and use the predictions 115 to improve the quality of patient care.

The medical device server 110, and the communication between the medical care facility 102 and the medical device server 110, can be secured by a variety of technologies. For example, in some embodiments, the medical device server 110 is secured via an API key and secret. The external facilities or devices that are authorized to communicate with the medical device server 110, such as the medical care facility 102, can be issued their unique key and secret. When an external device calls the medical device server 110, it can provide the key and the secret as part of an authorization header in the call. The medical device server 110 can validate the key and the secret to authorize the external calls before access and services are provided.

The requests 112 can include patient data and patient health data. In some embodiments, the requests 112 can include a field for a type of diagnosis the provider 106 desires to obtain by sending a request 112. For example, the provider can send a request 112 to request a diagnosis of sepsis or COVID. In other embodiments, the requests 112 may not include an explicit request for prediction of a type of disease or condition; instead, the medical device server 110, and the AI medical devices 114 therein, can automatically determine applicable AI medical devices 114, deploy them with the patient data embedded in the requests 112, and provide a prediction of a disease or condition corresponding to the deployed AI medical devices 114. In other words, in some embodiments, the provider 106 need not specify what type of condition or disease the provider 106 is suspecting. As a result, the environment 100 can be advantageous for early detection of diseases and conditions that are difficult to detect when they are in development phase versus when those conditions are fully presenting in a patient (sepsis is an example of the type of disease that is difficult to detect when in the development phase, but is relatively easy to detect once the patient has advanced in the development of sepsis).

The requests 112 can include a variety of information depending on the design and implementation of the environment 100. Any patient data, and/or patient health data, that are available and can be applicable to AI detection of a disease or condition can be included. For example, various timing data can be relevant to medical diagnosis and detection. The AI medical devices 114 can be configured to accept data having a selected range of timing to avoid or minimize the chance of using stale data. The medical device server 110 can include a manager 116 to handle various operations and functionality of the medical device server 110. In some examples of the environment 100, the manager 116 can enforce a global timing data restriction, whereby if patient data in a request is outside a selected range, the manager 116 does not call any AI medical device 114 and denies the request, sending along a corresponding message or status code, indicating the patient data in the request is stale. In some embodiments of the environment 100, the functionality of the manager 116 can be integrated into the AI medical devices 114, where a manager component of these devices can enforce timing and other specification of each device.

The requests 112 can include patient identification and medical information (PIMI), such as patient name, gender, age, ethnicity, current prescription, patient history and patient family history of any diseases or conditions that can be relevant to a prediction of a disease or health condition of the patient. PIMI and any other patient data in the requests 112 can have relevance to AI prediction of diseases and conditions. For example, the manager 116 can exclude deployment of a subset of the AI medical devices 114, based on patient age obtained from PIMI. The requests 112 can include patient visit data, including timing and initial physical examination data. These can include the date and time the patient visited the medical care facility 102, the patient's description of the reason for visit, the observations of the provider 106 upon initial physical examination or monitoring of the patient. Patient data and patient health data can become input features for one or more AI medical devices 114.

A request 112 can also include any lab orders, and lab observations, along with timing data for each. Orders can include a variety of blood tests, liver function tests, or any other medical tests. Observations can include the results of the medical tests. A request 112 can also include the patient's vitals data, such as blood pressure data, pulse rate, respiratory rate, body temperature, blood oxygen level, body mass index, the timing data of when the vitals were obtained and other measurements as may be obtained by a medical care facility 102 and/or the provider 106. The requests 112 can be in a variety of formats depending on the implementation of the environment 100. In one example, the requests 112 can be in JSON format.

The AI medical devices 114 can include various types in terms of their input/output features, and their internal operations. For example, some AI medical devices 114 can be rule-based, providing predictions based on rules. The AI medical devices 114 can include one or more AI models that can generate a numerical prediction of a disease or condition. The AI medical devices 114 can include output modules that map a numerical prediction to human-readable text output, based on rules, including selected ranges and thresholds. For example, in an AI medical device 114 trained to predict sepsis, an internal AI model may return a yProba classification in the form of a number between “0” to “1”. An output module can map the returned numerical prediction to a selected human-readable text. For example, for yProba classification values greater than “0.3”, a corresponding message can be generated that states that the patient can be at high risk of developing sepsis. For 0.05<yProba≤0.3, a corresponding message can be generated that states the patient is at moderate risk of sepsis. For yProba≤0.05, a corresponding message can be generated that states the patient is at low risk of developing sepsis. The generated messages can be routed to the medical care facility 102 as part of predictions 115. Similar to the sepsis AI medical device 114, other medical devices 114 can include rules to map the output of their AI models to human-readable text. Some AI medical devices 114 can, additionally or instead, include secondary numerical or non-numerical outputs. For example, toxicology-based AI medical devices 114 can render both a text output and a prescription dose recommendation or prediction. Such toxicology-based model AI medical devices 114 can also recommend a change to an existing prescription, based on current prescription data received in the request 112 and the output of a toxicology-based AI model.

The AI medical devices 114 can be AI models trained to receive a set of input values and provide a prediction of a disease or health condition. In some embodiments, each AI medical device 114 can include required and optional input fields, as well as required ranges of the input field values, based on the type of disease or condition that the model is trained to predict. For example, to predict a medical condition such as sepsis, various blood tests, as well as patient vitals data, may be required. The manager 116 can parse the incoming data in a request 112 and determine whether required input fields are present in the incoming data before deploying an AI medical device 114. In some embodiments, the manager 116 can determine an AI medical device 114 whose input fields match the type and nature of the incoming data in the request 112. The manager 116 can deploy the matched AI medical device 114 and provide the incoming data in the request to the input fields of the matched AI medical device 114.

In some embodiments, an AI medical device 114 can include a machine learning model, trained to classify the patient data in the request. In these embodiments, the machine learning model in an AI medical device 114 can be trained with a classification system, where a positive group training data and a negative group training data are used. For an AI medical device 114, which includes a machine learning model, trained to detect, or predict a health condition or disease of interest, the positive group training data includes patient data obtained from a patient population who have received a positive diagnosis of the condition or disease of interest. The negative group training data includes patient data obtained from a patient population who have not received a positive diagnosis or have received a negative diagnosis of the condition or disease of interest.

Training of the machine learning model can further include iteratively training the machine learning model with the positive and/or negative groups training data as sets of input features to the machine learning model. For each set of input features, the performance of the machine learning model can be assessed (e.g., by using AUC). The sets of input features that do not improve the performance of the machine learning model can be excluded from the machine learning model, thereby improving the performance of the machine learning model in detecting and/or predicting the condition or disease of interest.

In some embodiments, recursive feature elimination with cross-validation (RFECV) can be used to train the AI medical devices 114. An AI model in an AI medical device 114 can be iteratively trained on different sets of input features, where the model performance can be calculated for each set of input features. Input features that do not improve the model performance can be eliminated from subsequent training iterations. In one example, the gradient boosting model as implemented in XGBoost can be used. Example sets of input features used in the gradient boosting can include blood tests, such as BMP, CBC, FLT. The area under the curve (AUC) metric as described in the priority document of the present application can be used to assess the model performance for a set of input features.

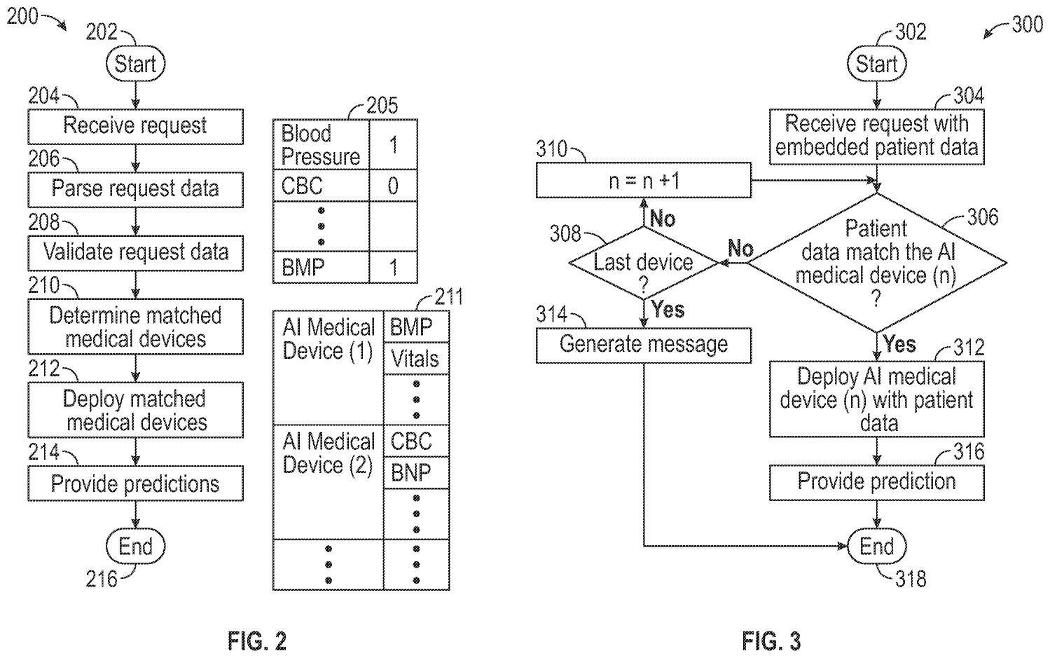

FIG. 2 illustrates a flowchart of a method 200 of the operations of the environment 100. The method starts at step 202. At step 204, a request 112 is received. The request 112 can be issued from a medical care facility 102 to the medical device server 110. The request 112 can include various patient data, such as PIMI, laboratory orders, observations, and timing data, as well as patient vitals. However, the requests 112 can vary greatly in the amount and type of patient data that they contain. The described embodiments of the medical device server 110 can flexibly be used, and a provider 106 can continuously utilize the AI medical devices 114, as more patient health data becomes available.

At step 206, the manager 116 parses the request 112 in relation to the input fields of the AI medical devices 114. For example, the manager 116 can generate a request table 205 of the type of data provided in the request. In one embodiment, the request table 205 can include a first column indicating a code for a type of patient data. In some embodiments, the first column in the request table 205 includes a list of all patient data, which can become input data to an input field of an AI medical device 114. The second column can indicate whether the request 112 includes the type of patient data indicated in the first column. For example, a first column can list codes or abbreviations for various patient data, including for example, laboratory orders, laboratory observations, patient vitals, and timing data of each. The second column can include a “1” for when the data is included in the request, or “0” for when the data is not included in the request.

At step 208, the manager 116 can validate the patient data in the request. Validation can depend on the type of data, and expected formats of a type of data. For example, for timing data, the manager 116 can determine whether dates and times are provided in the request with sufficient granularity to be useful as input features in the AI medical devices 114. Furthermore, validation can include checking the patient health data in a request against an expected range of values. Out of bound data may fail the validation step and/or cause the manager 116 to issue a warning message to the provider 106.

At step 210, the manager 116 can determine AI medical devices 114 whose input fields match the request data. For example, in one embodiment, the manager 116 can maintain an input fields table 211 of required and optional input fields of each AI medical devices 114. The input fields table 211 can be matched against the request table 205 to determine the AI medical devices 114 whose required and/or optional input fields data can be found in the request 112. At step 212, the manager 116 deploys the matched AI medical devices 114 and provides them with their required and optional input data. At step 214, the deployed AI medical devices 114 can provide their respective predictions 115, which are routed to the medical care facility 102 and/or the provider 106. The method ends at step 216.

FIG. 3 illustrates a flowchart of an example method 300 of the operations of the medical device server 110. In this example, the medical device server 110 includes AI medical devices 114 denoted by “AI medical devices(n),” where “n” can assume values “1” up to the number of AI medical devices 114 in the medical device server 110. The method starts at step 302. At step 304, a patient health prediction request 112 with embedded patient health data is received. At step 306, the manager 116 can determine whether the patient health data matches the required and/or the optional input fields of an AI medical device(n). If “No,” the method moves to step 308, where it is determined whether “n” in step 306 corresponded to the last AI medical device 114. If “No,” the method moves to step 310, where “n” is incremented by “1,” and the method returns to step 306. At step 308, if the AI medical device(n) in step 306 was the last AI medical device 114 in the medical device server 110, the method moves to step 314, where a message can be generated indicating that the patient health data in the request did not include the required fields for an AI medical device 114 to render a prediction. If at step 306, the manager 116 determines that the patient health data matches the required input fields of the AI medical device(n), the method moves to step 312, and the matched AI medical device(n) is deployed with the patient health data in the request provided as its input. At step 316, the matched AI medical device(n) provides a health prediction for the patient. The method ends at step 318.

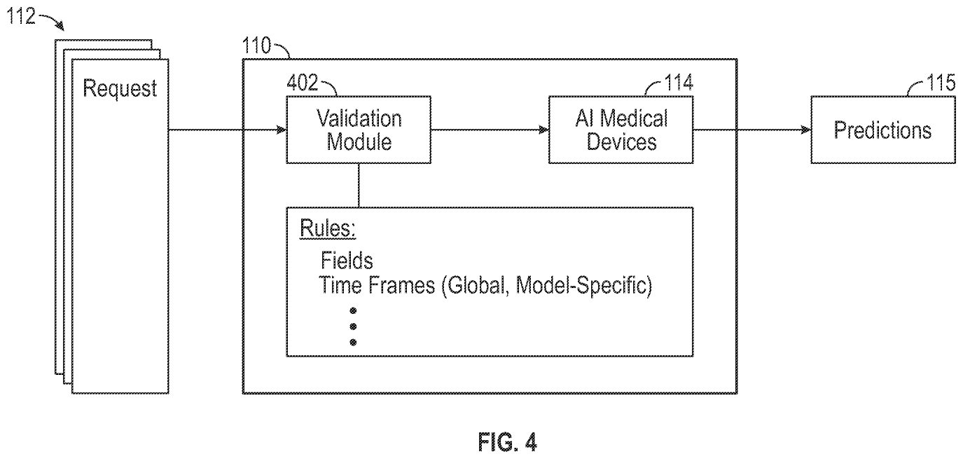

FIG. 4 illustrates a diagram of an embodiment, where the medical device server 110 can use a validation module 402 to improve the quality and accuracy of the predictions generated by the AI medical devices 114. The requests 112 can be received at the medical device server 110 by a validation module 402. In some embodiments, the validation module 402 can be implemented as part of the operations of the manager 116. In other embodiments, one or more validation modules 402 may be implemented as internal modules of the AI medical devices 114. In other embodiments, the validation module 402 can be an independent module. The validation module 402 can apply global and/or device-specific validation rules to accept or reject requests. The global and device-specific rules can depend on the input requirements of the AI medical devices 114. The validation module 402 can be configured to apply global validation rules applicable to requests directed to all AI medical devices 114. For example, in some embodiments, the validation module 402 can reject requests containing patient health data that are more than 24 hours old. In other embodiments, the validation module 402 can apply various timing requirement thresholds. For example, for some medical conditions, the time difference between the collection of patient sample and completion of the test cannot exceed a threshold (e.g., three hours). The validation module 402 can reject requests having patient health data falling outside of globally or individually selected thresholds. The term “global” in this context refers to validation rules applicable to all AI medical devices 114. The term “individual” in this context refers to validation rules applicable to a selection of the AI medical devices 114. The selection can include only one AI medical device 114 or more than one AI medical device 114.

The validation module 402 can be configured to apply validation rules applicable to an AI medical device 114. For example, the validation module 402 can determine whether the patient health data in a request is valid for an AI medical device 114. The determination of validation can include detecting the presence of the required and/or optional input fields data of the AI medical device 114 in the patient health data in the request, and also determining whether the patient health data in the request meets the selected ranges and thresholds of the input field data to be a valid input data for an AI medical device 114.

In some embodiments, the medical care facility 102 can send requests for health prediction, along with patient data, to the medical device server 110, on a rolling basis, as the facility generates various categories and types of patient health data. In this manner, the medical device server 110 can deploy applicable AI medical devices 114, based on available patient data, on a rolling basis. Some AI medical devices 114 require a combination of various tests and patient health measurement data before they can render a prediction. In these instances, a request missing the required input fields can be rejected by the validation module 402, until all the required input fields are present in the patient data in the request 112. For example, an AI medical device 114 may require CBC test data, B-type Natriuretic Peptide (BNP) test data and patient vitals to render a patient health prediction. In this scenario, an example first request 112 having only CBC test data will be rejected, with or without an accompanying status code or message. An example second request 112 having CBC test data and BNP test data will also be similarly rejected with an accompanying status code or message. An example third request 112, having CBC test data, BNP test data and the patient vitals data will pass the validation checks of the validation module 402. The third request 112 can be provided to an applicable AI medical device 114, and a patient health prediction 115 can be provided.

In some embodiments, each AI medical device 114 can have one or more validation rules. Example validation rules that can be implemented in a validation module 402 can include the following. Patient lab results must have been obtained in the first 24 hours of the patient visit. Lab results must be within three hours of the first lab result. In this scenario if both a CBC and a BMP are required, both tests should have been conducted within three hours of one another other (e.g., if the CBC resulted at 8:00 AM, then the BMP must have resulted by 11:00 AM the latest). Patient Age must be within a certain minimum and maximum age (e.g., 18 to 65). The required input fields data of an AI model of an AI medical device 114 must be present in the patient data in the request. An AI medical device 114 can have optional input data fields that may or may not be present in the patient data in a request. In the case of optional input data, none, some, or all of the optional input field data may be present in the patient data in the request, and a corresponding AI medical device 114 can nonetheless render a health prediction 115. For example, for a five-part differential blood test, some AI medical devices 114 require only that 1 of the 5 fields be present in the patient data, but do not require all five fields to render a health prediction. In this scenario, the validation module 402 can have a rule requiring at least one of the five parts of the five-part differential blood test and treating the remaining parts as optional. Some validation rules can be directed to units of measurement and/or format of the patient data in the request. For example, the validation module 402 can validate that the units of measurement for a test result matches an acceptable industry standard unit of measurement. Other validation rules can be directed to checking that the patient data in the request falls within selected ranges, and thresholds (e.g., minimum, and maximum values).

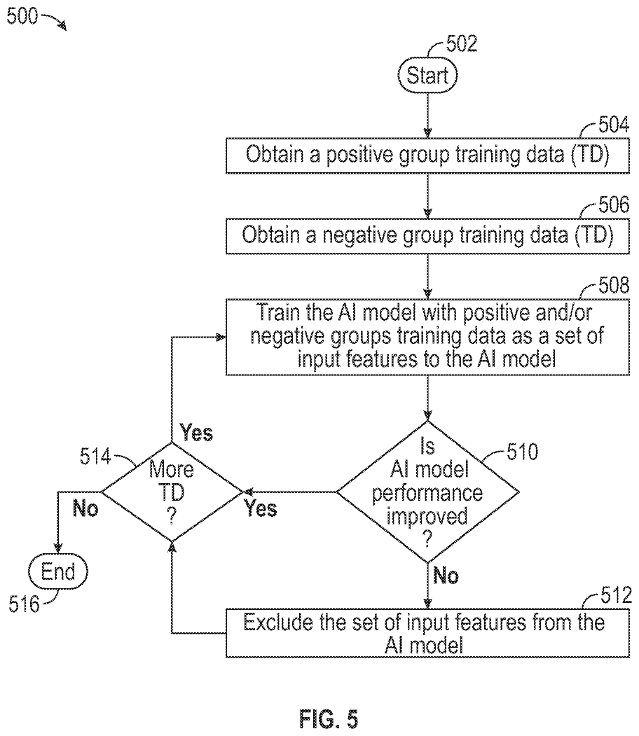

FIG. 5 illustrates a flowchart of a method 500 of training an artificial intelligence (AI) model (e.g., a machine learning model) in an AI medical device 114 to predict a disease or condition of interest. The method starts at step 502. At step 504, a positive group training data (TD) is obtained. The positive group TD includes patient data from a patient population, having received a positive diagnosis of the disease or condition of interest. At step 506, a negative group training data (TD) is obtained. The negative group TD includes patient data from a patient population who have not been diagnosed with the disease or condition of interest, or have been diagnosed not to have the disease or condition of interest. At step 508, the AI model is trained with the positive and/or negative groups training data as a set of input features to the AI model. At step 510 it is determined whether the performance of the AI model, using the set of input features of step 508, is improved.

At step 512, if the performance of the AI model using the set of the input features is not improved, the set of input features is excluded from the AI model. For example, it may be the case in some applications that from a universe of 29 blood test results, only 15 improve the performance of the AI model. The 14 blood test results that do not contribute to the improved performance of the AI model can be excluded from the model. In other words, the AI model, trained using the method 500 may not include input features corresponding to the 14 blood tests found not to be improving the performance of the AI model.

At step 510, if it is determined that the set of features improve the performance of the AI model, the method moves to step 514, where it is determined whether there is more training data to train the AI model. If yes, the method moves to step 508. If no, the method ends at step 516.

### Example Implementation Mechanism—Hardware Overview

Some embodiments are implemented by a computer system or a network of computer systems. A computer system may include a processor, a memory, and a non-transitory computer-readable medium. The memory and non-transitory medium may store instructions for performing methods, steps and techniques described herein.

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be server computers, cloud computing computers, desktop computer systems, portable computer systems, handheld devices, networking devices or any other device that incorporates hard-wired and/or program logic to implement the techniques.

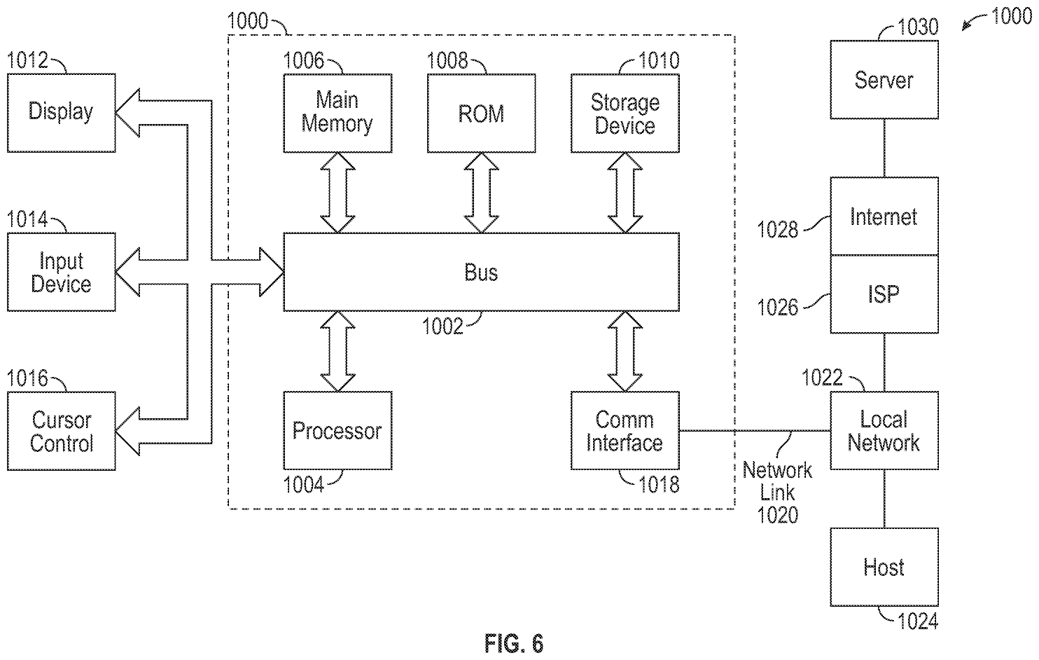

For example, FIG. 6 is a block diagram that illustrates a computer system 1000 upon which an embodiment of can be implemented. Computer system 1000 includes a bus 1002 or other communication mechanism for communicating information, and a hardware processor 1004 coupled with bus 1002 for processing information. Hardware processor 1004 may be, for example, special-purpose microprocessor optimized for handling audio and video streams generated, transmitted or received in video conferencing architectures.

Computer system 1000 also includes a main memory 1006, such as a random access memory (RAM) or other dynamic storage device, coupled to bus 1002 for storing information and instructions to be executed by processor 1004. Main memory 1006 also may be used for storing temporary variables or other intermediate information during execution of instructions to be executed by processor 1004. Such instructions, when stored in non-transitory storage media accessible to processor 1004, render computer system 1000 into a special-purpose machine that is customized to perform the operations specified in the instructions.

Computer system 1000 further includes a read only memory (ROM) 1008 or other static storage device coupled to bus 1002 for storing static information and instructions for processor 1004. A storage device 1010, such as a magnetic disk, optical disk, or solid state disk is provided and coupled to bus 1002 for storing information and instructions.

Computer system 1000 may be coupled via bus 1002 to a display 1012, such as a cathode ray tube (CRT), liquid crystal display (LCD), organic light-emitting diode (OLED), or a touchscreen for displaying information to a computer user. An input device 1014, including alphanumeric and other keys (e.g., in a touch screen display) is coupled to bus 1002 for communicating information and command selections to processor 1004. Another type of user input device is cursor control 1016, such as a mouse, a trackball, or cursor direction keys for communicating direction information and command selections to processor 1004 and for controlling cursor movement on display 1012. This input device typically has two degrees of freedom in two axes, a first axis (e.g., x) and a second axis (e.g., y), that allows the device to specify positions in a plane. In some embodiments, the user input device 1014 and/or the cursor control 1016 can be implemented in the display 1012 for example, via a touch-screen interface that serves as both output display and input device.

Computer system 1000 may implement the techniques described herein using customized hard-wired logic, one or more ASICs or FPGAs, firmware and/or program logic which in combination with the computer system causes or programs computer system 1000 to be a special-purpose machine. According to one embodiment, the techniques herein are performed by computer system 1000 in response to processor 1004 executing one or more sequences of one or more instructions contained in main memory 1006. Such instructions may be read into main memory 1006 from another storage medium, such as storage device 1010. Execution of the sequences of instructions contained in main memory 1006 causes processor 1004 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical, magnetic, and/or solid-state disks, such as storage device 1010. Volatile media includes dynamic memory, such as main memory 1006. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 1002. Transmission media can also take the form of acoustic or light waves, such as those generated during radio-wave and infra-red data communications.

Various forms of media may be involved in carrying one or more sequences of one or more instructions to processor 1004 for execution. For example, the instructions may initially be carried on a magnetic disk or solid state drive of a remote computer. The remote computer can load the instructions into its dynamic memory and send the instructions over a telephone line using a modem. A modem local to computer system 1000 can receive the data on the telephone line and use an infra-red transmitter to convert the data to an infra-red signal. An infra-red detector can receive the data carried in the infra-red signal and appropriate circuitry can place the data on bus 1002. Bus 1002 carries the data to main memory 1006, from which processor 1004 retrieves and executes the instructions. The instructions received by main memory 1006 may optionally be stored on storage device 1010 either before or after execution by processor 1004.

Computer system 1000 also includes a communication interface 1018 coupled to bus 1002. Communication interface 1018 provides a two-way data communication coupling to a network link 1020 that is connected to a local network 1022. For example, communication interface 1018 may be an integrated services digital network (ISDN) card, cable modem, satellite modem, or a modem to provide a data communication connection to a corresponding type of telephone line. As another example, communication interface 1018 may be a local area network (LAN) card to provide a data communication connection to a compatible LAN. Wireless links may also be implemented. In any such implementation, communication interface 1018 sends and receives electrical, electromagnetic or optical signals that carry digital data streams representing various types of information.

Network link 1020 typically provides data communication through one or more networks to other data devices. For example, network link 1020 may provide a connection through local network 1022 to a host computer 1024 or to data equipment operated by an Internet Service Provider (ISP) 1026. ISP 1026 in turn provides data communication services through the world wide packet data communication network now commonly referred to as the “Internet” 1028. Local network 1022 and Internet 1028 both use electrical, electromagnetic or optical signals that carry digital data streams. The signals through the various networks and the signals on network link 1020 and through communication interface 1018, which carry the digital data to and from computer system 1000, are example forms of transmission media.

Computer system 1000 can send messages and receive data, including program code, through the network(s), network link 1020 and communication interface 1018. In the Internet example, a server 1030 might transmit a requested code for an application program through Internet 1028, ISP 1026, local network 1022 and communication interface 1018.

The received code may be executed by processor 1004 as it is received, and/or stored in storage device 1010, or other non-volatile storage for later execution.

### EXAMPLES

It will be appreciated that the present disclosure may include any one and up to all of the following examples.

### Example 1

A method comprising: receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals; determining from a plurality of health prediction artificial intelligence medical devices, one or more health prediction artificial intelligence medical devices having input fields matching the patient data in the request; deploying the matched health prediction artificial intelligence medical devices with the patient data in the request; and providing one or more patient health predictions.

### Example 2

The method of Example 1, wherein a health prediction artificial intelligence medical device comprises a machine learning model, wherein the machine learning model is trained to classify the patient data and provide a patient health prediction, wherein training comprises: obtaining a positive group training data from a patient population who have received a positive diagnosis of a disease or condition of interest; obtaining a negative group training data from a patient population who have not received a positive diagnosis of the disease or condition of interest; iteratively training the machine learning model with the positive and negative groups training data as sets of input features to the machine learning model; determining the machine learning model performance for each set of input features; and excluding, from the machine learning model, sets of input features that do not improve performance of the machine learning model.

### Example 3

The method of some or all of Examples 1 and 2, wherein the request comprises an API request and the patient data comprises one or more of: BMP, liver function test (LFT), and CBC with differential.

### Example 4

The method of some or all of Examples 1-3, wherein the input fields of the health prediction artificial intelligence medical devices have a selected set of values and determining the matched health prediction artificial intelligence medical devices comprises determining whether the patient data in the request comprises the selected set of values.

### Example 5

The method of some or all of Examples 1-4, wherein the input fields of the health prediction artificial intelligence medical devices have a selected set of values and determining the matched health prediction artificial intelligence medical devices comprises determining whether the patient data comprises the selected set of values, wherein the method further comprises: when determining the patient data does not comprise the selected set of values, responding to the request with a status code indicating the missing selected set of values.

### Example 6

The method of some or all of Examples 1-5, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

### Example 7

The method of some or all of Examples 1-6, wherein the selected ranges comprise adult age ranges, pediatric age ranges, patient visit timing data, and patient laboratory observation timing data.

### Example 8

A non-transitory computer storage that stores executable program instructions that, when executed by one or more computing devices, configure the one or more computing devices to perform operations comprising: receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals; determining from a plurality of health prediction artificial intelligence medical devices, one or more health prediction artificial intelligence medical devices having input fields matching the patient data in the request; deploying the matched health prediction artificial intelligence medical devices with the patient data in the request; and providing one or more patient health predictions.

### Example 9

The non-transitory computer storage of Example 8, wherein a health prediction artificial intelligence medical device comprises a machine learning model, wherein the machine learning model is trained to classify the patient data and provide a patient health prediction, wherein training comprises: obtaining a positive group training data from a patient population who have received a positive diagnosis of a disease or condition of interest; obtaining a negative group training data from a patient population who have not received a positive diagnosis of the disease or condition of interest; iteratively training the machine learning model with the positive and negative groups training data as sets of input features to the machine learning model; determining the machine learning model performance for each set of input features; and excluding, from the machine learning model, sets of input features that do not improve performance of the machine learning model.

### Example 10

The non-transitory computer storage of some or all of Example 8 and 9, wherein the request comprises an API request and the patient data comprises one or more of: BMP, liver function test (LFT), and CBC with differential.

### Example 11

The non-transitory computer storage of some or all of Examples 8-10, wherein the input fields of the health prediction artificial intelligence medical devices have a selected set of values and determining the matched health prediction artificial intelligence medical devices comprises determining whether the patient data in the request comprises the selected set of values.

### Example 12

The non-transitory computer storage of some or all of Examples 8-11, wherein the input fields of the health prediction artificial intelligence medical devices have a selected set of values and determining the matched health prediction artificial intelligence medical devices comprises determining whether the patient data comprises the selected set of values, wherein the operations further comprise: when determining the patient data does not comprise the selected set of values, responding to the request with a status code indicating the missing selected set of values.

### Example 13

The non-transitory computer storage of some or all of Examples 8-12, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

### Example 14

The non-transitory computer storage of some or all of Examples 8-13, wherein the selected ranges comprise adult age ranges, pediatric age ranges, patient visit timing data, and patient laboratory observation timing data.

### Example 15

A system comprising a processor, the processor configured to perform operations comprising: receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals; determining from a plurality of health prediction artificial intelligence medical devices, one or more health prediction artificial intelligence medical devices having input fields matching the patient data in the request; deploying the matched health prediction artificial intelligence medical devices with the patient data in the request; and providing one or more patient health predictions.

### Example 16

The system of Example 15, wherein a health prediction artificial intelligence medical device comprises a machine learning model, wherein the machine learning model is trained to classify the patient data and provide a patient health prediction, wherein training comprises: obtaining a positive group training data from a patient population who have received a positive diagnosis of a disease or condition of interest; obtaining a negative group training data from a patient population who have not received a positive diagnosis of the disease or condition of interest; iteratively training the machine learning model with the positive and negative groups training data as sets of input features to the machine learning model; determining the machine learning model performance for each set of input features; and excluding, from the machine learning model, sets of input features that do not improve performance of the machine learning model.

### Example 17

The system of some or all of Examples 15 and 16, wherein the request comprises an API request and the patient data comprises one or more of: BMP, liver function test (LFT), and CBC with differential.

### Example 18

The system of some or all of Examples 15-17, wherein the input fields of the health prediction artificial intelligence medical devices have a selected set of values and determining the matched health prediction artificial intelligence medical devices comprises determining whether the patient data in the request comprises the selected set of values.

### Example 19

The system of some or all of Examples 15-18, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

### Example 20

The system of some or all of Examples 1-19, wherein the selected ranges comprise adult age ranges, pediatric age ranges, patient visit timing data, and patient laboratory observation timing data.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed is:

1. A method comprising:
receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals;
determining from a plurality of health prediction artificial intelligence medical devices, one or more health prediction artificial intelligence medical devices having input fields matching the patient data in the request;
deploying the matched health prediction artificial intelligence medical devices with the patient data in the request; and
providing one or more patient health predictions.

2. The method of claim 1, wherein a health prediction artificial intelligence medical device comprises a machine learning model, wherein the machine learning model is trained to classify the patient data and provide a patient health prediction, wherein training comprises:
obtaining a positive group training data from a patient population who have received a positive diagnosis of a disease or condition of interest;
obtaining a negative group training data from a patient population who have not received a positive diagnosis of the disease or condition of interest;
iteratively training the machine learning model with the positive and negative groups training data as sets of input features to the machine learning model;
determining the machine learning model performance for each set of input features; and
excluding, from the machine learning model, sets of input features that do not improve performance of the machine learning model.

3. The method of claim 1, wherein the request comprises an API request and the patient data comprises one or more of: BMP, liver function test (LFT), and CBC with differential.

4. The method of claim 1, wherein the input fields of the health prediction artificial intelligence medical devices have a selected set of values and determining the matched health prediction artificial intelligence medical devices comprises determining whether the patient data in the request comprises the selected set of values.

5. The method of claim 1, wherein the input fields of the health prediction artificial intelligence medical devices have a selected set of values and determining the matched health prediction artificial intelligence medical devices comprises determining whether the patient data comprises the selected set of values, wherein the method further comprises:
when determining the patient data does not comprise the selected set of values, responding to the request with a status code indicating the missing selected set of values.

6. The method of claim 1, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

7. The method of claim 6, wherein the selected ranges comprise adult age ranges, pediatric age ranges, patient visit timing data, and patient laboratory observation timing data.

8. A non-transitory computer storage that stores executable program instructions that, when executed by one or more computing devices, configure the one or more computing devices to perform operations comprising:
receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals;
determining from a plurality of health prediction artificial intelligence medical devices, one or more health prediction artificial intelligence medical devices having input fields matching the patient data in the request;
deploying the matched health prediction artificial intelligence medical devices with the patient data in the request; and
providing one or more patient health predictions.

9. The non-transitory computer storage of claim 8, wherein a health prediction artificial intelligence medical device comprises a machine learning model, wherein the machine learning model is trained to classify the patient data and provide a patient health prediction, wherein training comprises:
obtaining a positive group training data from a patient population who have received a positive diagnosis of a disease or condition of interest;
obtaining a negative group training data from a patient population who have not received a positive diagnosis of the disease or condition of interest;
iteratively training the machine learning model with the positive and negative groups training data as sets of input features to the machine learning model;
determining the machine learning model performance for each set of input features; and
excluding, from the machine learning model, sets of input features that do not improve performance of the machine learning model.

10. The non-transitory computer storage of claim 8, wherein the request comprises an API request and the patient data comprises one or more of: BMP, liver function test (LFT), and CBC with differential.

11. The non-transitory computer storage of claim 8, wherein the input fields of the health prediction artificial intelligence medical devices have a selected set of values and determining the matched health prediction artificial intelligence medical devices comprises determining whether the patient data in the request comprises the selected set of values.

12. The non-transitory computer storage of claim 8, wherein the input fields of the health prediction artificial intelligence medical devices have a selected set of values and determining the matched health prediction artificial intelligence medical devices comprises determining whether the patient data comprises the selected set of values, wherein the operations further comprise:
when determining the patient data does not comprise the selected set of values, responding to the request with a status code indicating the missing selected set of values.

13. The non-transitory computer storage of claim 8, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

14. The non-transitory computer storage of claim 13, wherein the selected ranges comprise adult age ranges, pediatric age ranges, patient visit timing data, and patient laboratory observation timing data.

15. A system comprising a processor, the processor configured to perform operations comprising:
receiving a patient health prediction request, the request comprising patient data, the patient data comprising one or more of patient identification, patient visit timing data, patient laboratory order data, patient laboratory order timing data, patient laboratory observation data, patient laboratory observation timing data, and patient vitals;
determining from a plurality of health prediction artificial intelligence medical devices, one or more health prediction artificial intelligence medical devices having input fields matching the patient data in the request;
deploying the matched health prediction artificial intelligence medical devices with the patient data in the request; and
providing one or more patient health predictions.

16. The system of claim 15, wherein a health prediction artificial intelligence medical device comprises a machine learning model, wherein the machine learning model is trained to classify the patient data and provide a patient health prediction, wherein training comprises:
obtaining a positive group training data from a patient population who have received a positive diagnosis of a disease or condition of interest;
obtaining a negative group training data from a patient population who have not received a positive diagnosis of the disease or condition of interest;
iteratively training the machine learning model with the positive and negative groups training data as sets of input features to the machine learning model;
determining the machine learning model performance for each set of input features; and
excluding, from the machine learning model, sets of input features that do not improve performance of the machine learning model.

17. The system of claim 15, wherein the request comprises an API request and the patient data comprises one or more of: BMP, liver function test (LFT), and CBC with differential.

18. The system of claim 15, wherein the input fields of the health prediction artificial intelligence medical devices have a selected set of values and determining the matched health prediction artificial intelligence medical devices comprises determining whether the patient data in the request comprises the selected set of values.

19. The system of claim 15, wherein the input fields of the health prediction artificial intelligence medical devices comprise selected values and corresponding selected ranges of the selected values, and determining the matched health prediction artificial intelligence devices comprise determining whether patient data comprises the selected values and the corresponding selected ranges for a health prediction artificial intelligence medical device.

20. The system of claim 19, wherein the selected ranges comprise adult age ranges, pediatric age ranges, patient visit timing data, and patient laboratory observation timing data.
